(19) United States
(12) Patent Application Publication
(10) Pub. No.: US 2016/0220589 A1
(43) Pub. Date: Aug. 4, 2016

(54) COMPOSITIONS AND METHODS FOR THE TREATMENT OF SKIN DISORDERS

(71) Applicant: DERMIPSOR LTD., Rehovot (IL)

(72) Inventors: Avikam HAREL, Tel Aviv (IL); Olga BLOCH, Petach Tikva (IL)

(21) Appl. No.: 15/097,938

(22) Filed: Apr. 13, 2016

Related U.S. Application Data
(62) Division of application No. 12/242,243, filed on Sep. 30, 2008, now abandoned.
Application No. 12/242,243 is a continuation-in-part of application No. 10/192,842, filed on Jul. 11, 2002, now abandoned.
Application No. 10/192,842 is a continuation-in-part of application No. PCT/IL01/00017, filed on Jan. 9, 2001.
Application No. 12/242,243 is a continuation-in-part of application No. 11/914,093, filed on Jun. 10, 2008, now Pat. No. 8,034,788.
Application No. 12/242,243 is a continuation-in-part of application No. 11/914,102, filed on Jun. 10, 2008, now Pat. No. 9,173,835.
(60) Provisional application No. 60/679,329, filed on May 10, 2005.
(60) Provisional application No. 60/679,556, filed on May 10, 2005.

(30) Foreign Application Priority Data
Jan. 11, 2000 (IL) 133976

(51) Int. Cl. A61K 31/593; A61K 31/455

(52) U.S. Cl. CPC A61K 31/593; A61K 31/455

(57) ABSTRACT

Methods and pharmaceutical compositions for use in the treatment of benign or malignant proliferative pathologies are disclosed. The methods involve the topical administration of nicotinamide or a nicotinamide derivative in combination with a vitamin D compound. The combination is shown to act in synergy and is especially useful for treatment of psoriasis and other skin disorders.

### CROSS-REFERENCE TO RELATED APPLICATIONS

This application is a division of application Ser. No. 12/242,243 filed Sep. 30, 2008, which is a continuation-in-part of application Ser. No. 10/192,842 filed Jul. 11, 2002, now abandoned, which is a continuation-in-part of International application no. PCT/IL01/00017 filed Jan. 9, 2001, which claims priority to Israel application no. 133976 filed Jan. 11, 2000. application Ser. No. 12/242,243 is also a continuation-in-part of application Ser. No. 11/914,093 filed Jun. 10, 2008, now U.S. Pat. No. 8,034,788, which is the 371 filing of PCT/IL2006/000552 filed May 10, 2006, which claims the benefit of application No. 60/679,329 filed May 10, 2005. application Ser. No. 12/242,243 is also a continuation-in-part of application Ser. No. 11/914,102 filed Jun. 10, 2008, now U.S. Pat. No. 9,173,835, which is the 371 filing of PCT/IL2006/000553 filed May 10, 2006, which claims the benefit of application No. 60/679,556 filed May 10, 2005. The entire content of each prior application is expressly incorporated herein by reference thereto.

### FIELD OF THE INVENTION

The present invention relates to compositions and methods for treating skin disorders and, more particularly, for treating hyperproliferative epidermal pathologies and other conditions of the epidermis.

### BACKGROUND

### Hyperproliferative Skin Disorders and Diseases

Hyperproliferative skin disorders refers to diseases and disorders, which are characterized by a higher than normal level of proliferation of epidermal cells known as keratinocytes, and, as a rule, also by abnormal differentiation of epidermal cells.

The hyperproliferative epidermal pathology may be malignant or benign. Malignant epidermal pathologies include: squamous-cell carcinoma (SCC), basal-cell carcinoma (BCC) and other non-melanoma skin cancers (NMSCs). Representative examples of benign epidermal pathologies include psoriasis, common warts, keratoacanthoma, seborrhoeic keratosis, seborrhea and ichthyosis.

Normal growth and differentiation of epidermal cells requires a number of regulating factors such as Vitamin D3, Vitamin A, a number of cytokines and growth factors and extra- and intracellular free Ca⁺².

Skin benign and malignant hyperproliferative disorders arise from faulty regulation of growth and differentiation of epidermal cells. The faulty regulation is often caused by lack of appropriate response to regulating factors, or by abnormal levels or function of the regulating factors themselves. For example, it is well known that inappropriate growth and differentiation of epidermal cells results from aberrant signaling through the epidermal growth factor receptor. This abnormality may contribute to the development of various skin disorders such as psoriasis, ichythyiosis, squamous cell carcinomas and multiple human tumors. Hence, controlling the differentiation and/or proliferation of epidermal cells is advantageous in treating hyperproliferative skin disorders.

Psoriasis is caused by unknown factors that stimulate T-lymphocyte activation, proliferation, and cytokine release that leads to hyperproliferation of keratinocytes. Although the etiology of psoriasis is unknown, the affected keratinocytes are responsible for the typical clinical features of the disease: well-demarcated inflamed skin lesions covered with a silvery white scale, covering about 10%-15% of the body surface. Topical administration of therapeutic cream or ointment is used for treating mild and moderate cases, while phototherapy requires repeated visits to the clinic and exposes the patient to the concomitant dangers of radiation. Systemic treatment, relying on immunosuppressive therapy, is limited to severe cases due to its serious side effects.

### Vitamin D and Hyperproliferative Skin Disease

Vitamin D is a prohormone with several active metabolites that act as hormones. In the skin, previtamin D3 is synthesized photochemically from 7-dehydrocholesterol and is slowly isomerized to vitamin D3, which is removed by vitamin D-binding protein. In the liver, vitamin D3 is converted to 25(OH)D3, the major circulating form, which passes through the enterohepatic circulation and is reabsorbed from the gut. In the kidneys, it is further hydroxylated to the more metabolically active form, 1α,25(OH)₂D3, also termed: calcitriol, 1α,25-dihydroxycholecalciferol, and vitamin D hormone.

Experimental evidence has shown that vitamin D and its analogs function as anti-proliferative agents and stimulate the terminal differentiation of keratinocytes.

Vitamin D and certain analogs are effective in treating the hyperprofilerated state shown in psoriasis vulgaris (reviewed in DeLuca 1988; Lehmann et al., 2004). The systemic administration of these compounds is limited by their toxicity and adverse effect on calcium metabolism, therefore topical preparations are preferred.

Calcipotriol (calcipotriene), a synthetic vitamin D3 analog, is marketed in the United States as a topical antipsoriatic under the trade name DOVONEX® (USA) or DOVONEX® (Europe). Calcipotriol is as potent as the naturally occurring calcitriol in regulating cell proliferation, but has the benefit of being much less active in its effect on calcium metabolism. Despite this, calcipotriol is only partially effective in treating psoriatic lesions.

The chemical structures of calcipotriol and of calcitriol are illustrated in FIG. 1.

The art provides some examples of vitamin D analogs and derivatives and compositions comprising the same: U.S. Pat. No. 4,851,401 (cyclopentano-vitamin D analogs), U.S. Pat. No. 5,120,722 (trihydroxycalciferol derivatives), U.S. Pat. No. 5,446,035 (20-methyl substituted vitamin D), U.S. Pat. No. 5,411,949 (23-oxa-derivatives), U.S. Pat. No. 5,237,110 (19-nor-vitamin D compounds), U.S. Pat. No. 4,857,518 (hydroxylated 24-homo-vitamin D derivatives). Additional Vitamin D analogs are taught in U.S. Pat. Nos. 4,804,502; 4,866,048; 5,145,846 5,374,629; 5,403,940; 5,446,034; and 5,447,924, 7,115,758, 7,312,249, 7,241,752, 7,361,664, among others.

U.S. Pat. No. 5,037,816 is directed to treating psoriasis by topically administering a vitamin D compound. U.S. Pat. No. 5,194,248 similarly describes topical administration of a vitamin D analog. U.S. Pat. No. 6,552,009 discloses a composition comprising a vitamin D compound and a derivative of retinoid, for treating abnormal cell-proliferation. The vitamin D compound may be calcitriol or calcipotriol.

U.S. Pat. No. 6,753,013 and U.S. Pat. No. 6,787,529 describe a pharmaceutical composition for dermal use comprising a combination of a vitamin D compound and a corticosteroid, the composition alleviating the inconvenience of a two-component regimen for the treatment of psoriasis and other inflammatory skin diseases.

There are have been many reports of treatment of psoriasis and other related skin disorders in humans, following oral or topical treatment with vitamin D3 compounds [1]. However, response to treatment varies among individuals, leading to a continued search for novel compositions that will alleviate symptoms in the majority of patients.

At present, the most potent topical treatment available for psoriasis is the combination of calcipotriol and a corticosteroid (e.g. DAIVOBET® and TACLONEX®). This treatment involves hazards generally associated with administration of corticosteroids, such as leaching of the drug into the bloodstream, leading to immune suppression, Cushing's syndrome, weight gain, growth retardation, etc.

Existing topical treatment regimens force patients and physicians to choose between efficacy and safety, as none of the current treatments combines both. The need exists for a treatment regimen that alleviates the need for steroid administration. There is a clear medical need and market, for a new topical treatment that simultaneously offers significant efficacy and a superior safety profile.

### Nicotinamide

Nicotinamide (NA, niacinamide), a derivative of vitamin B₃ and a precursor of the coenzyme nicotinamide adenine dinucleotide (NAD), displays multiple functions in cell metabolism. NA has been shown to induce the differentiation of insulin-producing cells (Otonkoski et al, 1993).

U.S. Pat. No. 6,248,763 relates to specific topical compositions for treating skin conditions for example acne and psoriasis, which comprise 0.01%-1% methyl nicotinate, as the active ingredient.

U.S. Pat. No. 4,505,896 discloses compositions and methods for the treatment of acne vulgaris, including use of nicotinic acid or nicotinamide and, optionally, another chemical agent known to be effective in treating acne. Nevertheless, the compositions of U.S. Pat. No. 4,505,896 are specifically directed toward the treatment of acne vulgaris, which is an inflammatory disease and not a hyperproliferative benign (e.g., psoriasis) or malignant skin disorders.

U.S. Pat. No. 6,248,763 discloses compositions for treating skin conditions, which include derivatives of nicotinic acid or nicotinamide and, in particular, methyl nicotinate, as the active ingredient. These compositions are topically applied and are directed toward the treatment of acne and other skin conditions such as fine lines and age spots, burns, etc. However, like U.S. Pat. No. 4,505,896, U.S. Pat. No. 6,248,763 fails to teach compositions and methods for the treatment of hyperproliferative skin disorders.

U.S. Pat. No. 4,067,975 to Yu describes a topical composition of 6-nicotinamide and its use for treating psoriasis; however, in contrast, nicotinamide and nicotinic acid are explicitly mentioned as being completely ineffective for this use.

US Statutory Invention Registration No. H2013 to Boyd et al., of the WO 98/52529 patent family, describes a specific skin care composition comprising nicotinamide among its other active ingredients. There is no experimental disclosure pertaining to use of this composition to treat hyperproliferative diseases, rather the publication repeatedly discusses prevention of skin aging. Exemplified are highly specific formulations which are aimed to hydrate the skin without causing dryness typically associated with nicotinamide application.

The background art does not teach topical administration of nicotinamide for treatment of benign or malignant hyperproliferative epidermal pathologies.

U.S. Pat. No. 6,107,349 to Mantynen, describes treatment of psoriasis by oral administration of a series of active agents including Vitamin E, folic acid, evening primrose oil, vitamin B compounds, and optionally fatty acids. The individual effect of each of these components was not tested, so that it is unclear which one is a true active ingredient; the Mantynen claims are limited to the entire combination. In addition, oral administration of pharmaceutical compositions is typically associated with considerable side effects, especially during long-term administration to a diverse population.

It would be desirable to develop a topical composition for application upon the skin of psoriasis patients, or for treatment of additional epidermal hyperproliferative diseases. The topical composition preferably should penetrate into the skin but should not enter the systemic circulation to the extent that would cause side effects frequently found in orally administered medications.

The background art does not suggest the novel combination of the present invention, of nicotinamide and a vitamin D compound for preparation of a topical composition for application upon the skin, useful in treating hyperproliferative epidermal pathologies. Nowhere in the background art is it taught or suggested that a Vitamin B3 derivative combined with a Vitamin D compound in a composition for topical administration would provide synergistic therapeutic benefit for treating epidermal pathologies.

### SUMMARY OF THE INVENTION

While conceiving the present invention, it was hypothesized that a vitamin B3 derivative, such as nicotinamide, when combined with a Vitamin D compound, could exert improved anti-proliferative effects in various epidermal cell associated pathologies.

While reducing the present invention to practice, it was found that (i) nicotinamide promotes the differentiation and inhibits the proliferation of benign and malignant epidermal cells; (ii) combinations of nicotinamide and Vitamin D compounds exert a synergistic effect on epidermal cell proliferation; and (iii) nicotinamide is highly effective as an anti-oxidant against auto-oxidative agents.

The present invention provides compositions comprising a vitamin D compound and a vitamin B3 derivative such as nicotinamide, and methods of use thereof for the treatment of benign or malignant hyperproliferative diseases of the skin. In particular, the inventors of the present invention have unexpectedly found that nicotinamide, when provided in combination with calcipotriol, produces a synergistic effect in reducing the symptoms of the hyperproliferative disease psoriasis. Synergistic effects were similarly seen in a mouse model of disease.

Accordingly, the present invention provides highly efficacious therapeutic compositions comprising calcipotriol and nicotinamide.

In the compositions of the present invention, the concentration of nicotinamide preferably ranges between 4.1 mM (0.05%) and 491.3 mM (6%).

In a certain currently preferred embodiment, the concentration of nicotinamide ranges between 14.7 mM (0.18%) and 147.4 mM (1.8%). The concentration of nicotinamide is based on a molecular weight of 122.13 for nicotinamide.

In one embodiment of the invention, the concentration of nicotinamide is 0.21% of the final composition, and the concentration of calcipotriol is 0.005% of the final composition.

In a certain currently preferred embodiment, the final concentration of nicotinamide is 1.4%, and the final concentration of calcipotriol is 0.005%

In certain embodiments, the concentration of the vitamin D compound is within the range of 0.001%-0.01% of the final composition. In one embodiment, the concentration is within the range of 0.003% to 0.007%, and in a currently preferred embodiment, the concentration is 0.005%.

Further, in certain embodiments of the present invention, the vitamin D compound may be selected from the following: vitamin D3, vitamin D2, 25(OH)D3, 1α,25(OH)₂D3, a 19-nor-vitamin D compound, a cyclopentano-vitamin D derivative, a trihydroxycalciferol derivative, a 20-methyl substituted vitamin D derivative, a 23-oxa-derivative, and a hydroxylated 24-homo-vitamin D derivative.

Moreover, in the present invention, the vitamin D compound may be a vitamin D metabolite, a vitamin D agonist, a vitamin D prodrug, a vitamin D derivative or a vitamin D analog.

Still further, the present invention comprises administration of the combination of a vitamin D compound, along with a vitamin B3 derivative selected from one of the following: nicotinamide, a nicotinamide analog or a nicotinamide derivative. Non-limiting examples of nicotinamide derivatives include 2-amino-nicotinamide derivatives, 5-phenyl-nicotinamide derivatives, and 6-substituted nicotinamide derivatives.

The composition of the invention may be administered in order to increase the anti-oxidative properties of epidermal cells. The composition may be administered to inhibit proliferation or promote the differentiation of epidermal cells in need thereof.

The pharmaceutical compositions of the present invention can optionally be packaged in a container and identified in print in or on the container, for use in the treatment of a benign and/or a malignant hyperproliferative epidermal pathology.

Other methods of increasing anti-oxidative properties of epidermal cells, according to the present invention, comprise contacting the cells with a composition comprising nicotinamide or a nicotinamide derivative, in combination with an effective amount of a Vitamin D compound. According to further features in preferred embodiments of the invention described below, the hyperproliferative benign epidermal pathology is selected from the group consisting of psoriasis, ichythyiosis, common warts, keratoacanthoma, seborrhoic keratosis and seborrhea.

According to still further features in the described preferred embodiments the hyperproliferative malignant epidermal pathology is selected from the group consisting of squamous-cell carcinoma (SCC), basal cell carcinoma (BCC) and a non-melanoma skin cancer (NMSC).

Moreover, in one embodiment, the composition of the invention consists essentially of: a) a vitamin B3 derivative selected from: nicotinamide, and a nicotinamide derivative; and b) a vitamin D compound. The composition further comprises a pharmaceutically acceptable carrier suitable for topical treatment of benign or malignant hyperproliferative epidermal diseases.

According to still further features in the described currently preferred embodiments the Vitamin D compound is calcipotriol. In other embodiments, the Vitamin D compound is 1α,25-dihydroxy-vitamin D3, also termed calcitriol.

According to still further features in the described embodiments, the concentration of the vitamin D compound ranges between 1 nM and 200 nM.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. In the drawings:

FIG. 1 illustrates the chemical structures of calcitriol, a vitamin D metabolite, and of calcipotriol, a currently preferred vitamin D analog.

FIG. 2A graphically illustrates the anti-proliferative effect of NA on immortalized human keratinocytes (HaCat) and on the human epithelial carcinoma cells (A431) cells. The MTT proliferation assay was used.

FIG. 2B graphically illustrates the anti-proliferative effect of NA on primary cultured human epidermal keratinocytes obtained from routine face-lift surgery cells (a primary cell line)

FIG. 3 graphically illustrates that administration of the vitamin D compound 1α 25(OH)₂D3 to HaCat and A431 cells did not effect the cell proliferation;

FIGS. 4A and 4B plot the anti-proliferative effect of a combination of NA and a D3 metabolite (1α 25(OH)₂D3) on HaCat cell line proliferation (FIG. 4A) and the synergistic effect of this combination as compared with the anti-proliferative effects of each of these compounds separately (NA and 1α 25(OH)₂D3), on this cell line, shown as the effect of the combined treatment minus the effect of each of the compounds (FIG. 4B);

FIGS. 5A and 5B graphically illustrate the anti-proliferative effect of a combination of NA and a D3 metabolite (1α 25(OH)₂D3) on A431 cell line proliferation (FIG. 5A) and the synergistic effect of this combination as compared with the anti-proliferative effects of each of these compounds separately (NA and 1α 25(OH)₂D3), on this cell line, shown as the effect of the combined treatment minus the effect of each of the compounds (FIG. 5B);

FIG. 6 shows the effect of NA on involucrin and keratin k10 expression in HaCat cells;

FIG. 7 shows the effect of NA on basal and envelope cornified cell expression in HaCat cell line;

FIG. 8 shows the effect of NA on apoptosis level in HaCat and A431 cell lines;

FIG. 9 shows the resistance of HaCat cells treated for long-term period with NA to oxidative stress induced by hydrogen peroxide (H₂O₂).

FIG. 10 illustrates the synergistic effect of the combination nicotinamide and calcipotriol, in topical administration in a mouse model of psoriasis.

FIG. 11 presents results of topical administration of the combination nicotinamide and calcipotriol upon psoriatic lesions, in a human phase II trial.

FIG. 12 illustrates the synergistic effect of the combination nicotinamide and calcipotriol, when applied topically to psoriatic lesions in a human phase II trial.

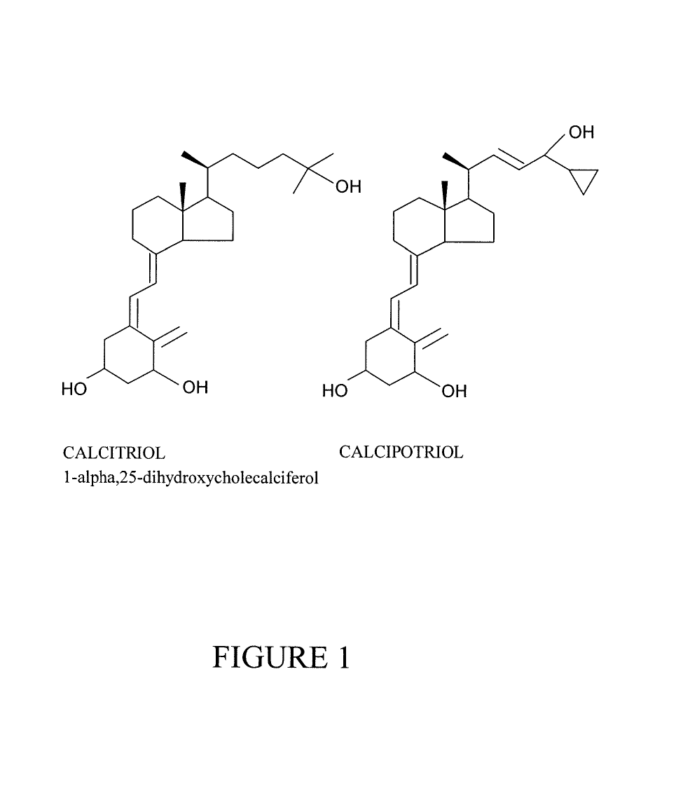

### DETAILED DESCRIPTION OF THE PREFERRED EMBODIMENTS

The present invention provides a pharmaceutical composition, and method, which can be used in the treatment of skin disorders. Specifically, the present invention can be used in the treatment of benign and malignant proliferative epidermal pathologies.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

While conceiving the present invention, it was hypothesized that a vitamin B3 derivative such as nicotinamide, when used in combination with a vitamin D compound, could result in enhanced anti-proliferative activity.

While reducing the present invention to practice, as is demonstrated in the Examples section that follows, it was found that nicotinamide in combination with a vitamin D compound, promotes the differentiation and inhibits the proliferation of benign and malignant epidermal cells. The combination of nicotinamide and a vitamin D compound acted synergistically, giving improved results over each of the active agents when used alone.

As is described in detail in the Examples section, the antiproliferative activity of these compounds was initially tested in three model systems:
(i) a spontaneously immortalized human keratinocyte line, which is referred to herein as “HaCat cell line” or “HaCat cells”. The HaCat cell line serves as a model for highly proliferative epidermis, such as, but not limited to, psoriatic epidermis [2]. The line also serves as a model for effects of external modulators of epidermal differentiation [3].
(ii) a human epidermal carcinoma cell line, which is referred to herein as “A431 cell line” or “A431 cells”, bearing the mutated alleles of p53. The A431 cell line serves as a model for testing anti-carcinogenic drugs [4] and hence as a model for malignant hyperproliferative pathologies.
(iii) rapidly proliferating human keratinocytes, which are referred to herein as “Primary Cultured Human Epidermal Keratinocytes”, and serves as a model for evaluating treatment of psoriasis [5].

A synergistic effect was then demonstrated in vivo, for the combination of nicotinamide and the vitamin D3 synthetic analog calcipotriol, in the following systems: A mouse-tail test which is standard for evaluating antipsoriatic activity of a tested treatment, and during phase II human trials on psoriatic lesions. These are discussed in greater detail hereinbelow in the Examples section.

The results demonstrated hereinbelow indicate that a composition comprising nicotinamide and a vitamin D compound as the active agents is highly potent and versatile for treatment of hyperproliferative epidermal pathologies.

Hence, according to the present invention, there are provided methods of treating benign or malignant hyperproliferative epidermal pathologies in a subject in need thereof. These methods are effected by administering to the subject a therapeutically effective amount of nicotinamide or a nicotinamide derivative, in combination with a vitamin D compound.

### GLOSSARY

As used herein, the phrase “hyperproliferative epidermal pathology” includes any disease, condition or syndrome that is characterized by a higher than normal level of proliferation of epidermal cells, and, as a rule, also by abnormal differentiation.

The hyperproliferative epidermal pathology may be malignant or benign, as is discussed hereinabove and is demonstrated in detail in the Examples section that follows.

Representative examples of malignant hyperproliferative epidermal pathologies that are treatable by the methods of the present invention include, without limitation, squamous-cell carcinoma (SCC), basal-cell carcinoma (BCC) and other non-melanoma skin cancers (NMSCs).

Representative examples of benign hyperproliferative epidermal pathologies that are treatable by the methods of the present invention include, without limitation, psoriasis, common warts, keratoacanthoma, seborrhoic keratosis, seborrhea and ichthyosis.

The term “vitamin D compound”, according to the present invention, refers to a molecule having a 9,10-seco-steroidal structure, and similar chemical or biological activity to vitamin D3. Included are naturally occurring vitamin D3 metabolites, synthetic derivatives, analogs, prodrugs, and vitamin D receptor agonists.

The term “metabolite” refers to a naturally occurring breakdown product.

The term “prodrug” refers to a compound that, upon administration, is activated in vivo into an active form, in one or more steps.

The term “derivative” refers to a synthetically modified compound which partially or exactly mimics the chemical or biological activity of the well-known parent compound, yet is not identical in chemical structure to the parent compound. A “vitamin D3 derivative” is a non-endogenous product of a chemical synthetic reaction using as a substrate any of the following: vitamin D3, a vitamin D3 precursor, a vitamin D metabolite or an analog thereof.

The term “analog” refers to a synthetically modified compound which partially or exactly mimics the chemical or biological activity of the well-known parent compound, yet is not identical in chemical structure to the parent compound. A “vitamin D3 analog” is a non-endogenous product of a chemical synthetic reaction which uses a substrate other than any of the naturally occurring following group: vitamin D3, a vitamin D metabolite, a vitamin D3 precursor. A non-limiting example of a vitamin D3 analog is calcipotriol.

In the art, the terms “analog” and “derivative” tend to be used interchangeably.

Non-limiting examples of vitamin D3 metabolites include 25(OH)D3, and calcitriol (also termed 1α,25(OH)₂D3, and vitamin D hormone).

Non-limiting examples of vitamin D3 prodrugs (or precursors) include 7-dehydrocholesterol, and previtamin D3. Synthetic prodrugs are also available, such that upon administration they are activated in vivo into an active form.

The term “vitamin D agonist” refers to a compound which interacts with the vitamin D receptor and induces at least some of the biological activities induced by the endogenous ligand 1α,25-dihydroxy-vitamin D3, but is other than α,25-dihydroxy-Vitamin D3. Such agonists are included in the scope of the invention. A non-limiting example of a vitamin D3 agonist is vitamin D2, a synthetic analog of vitamin D3, also termed “ergocalciferol”.

There are numerous synthetic Vitamin D3 analogs and derivatives in existence, some examples are mentioned the Background, and are incorporated herein by reference.

A definitive essay on well-known Vitamin D compounds, including analogs, derivatives and metabolites, as well as definitions of terminology, may be found in “Nomenclature of Vitamin D, Recommendations 1981”, issued by the International Union of Biochemistry and Molecular Biology, IUPAC-IUB Joint Commission on Biochemical Nomenclature (JCBN), incorporated herein by reference. Similarly, see “Vitamin D; Physiology, Molecular Biology and Clinical Applications” edited by Michael F. Holick, published in 1999 by Human Press, especially Chapter 4 by Glenville Jones, incorporated herein by reference.

Preferably, the vitamin D compound of the invention is selected from cholecalciferol, calcifediol, calcitriol and calcipotriol.

In a certain currently preferred embodiment, the vitamin D compound is calcipotriol (also termed “calcipotriene”).

Non-limiting examples of nicotinamide derivatives include 2-amino-nicotinamide derivatives (described in U.S. Pat. No. 6,624,174), 5-phenyl-nicotinamide derivatives (disclosed in WO 08031734) and 6-substituted nicotinamide derivatives (described in U.S. Pat. No. 7,399,774).

Herein, the term “treating” includes abrogating, substantially inhibiting, slowing or reversing the progression of a pathology, substantially ameliorating clinical symptoms of a pathology or substantially preventing the appearance of clinical symptoms of a pathology. These effects may be manifested, for example, by a decrease in the rate of proliferation, an improved differentiation or a combination thereof and/or by complete elimination of the abnormal proliferation and differentiation of the epidermal cells in the treated subject.

The term “administering” as used herein describes a method for bringing a nicotinamide agent, a vitamin D compound, or a combination of agents described herein, and epidermal cells affected by the pathology together in such a manner that the agent can affect the proliferation and/or differentiation of these cells.

The phrase “therapeutically effective amount”, as used herein, describes an amount administered to an individual, which is sufficient to abrogate, substantially inhibit, slow or reverse the progression of an epidermal pathology, to substantially ameliorate clinical symptoms of an epidermal pathology or substantially prevent the appearance of clinical symptoms of an epidermal pathology.

More specifically, the phrase “therapeutically effective amount” defined above, describes an amount of an agent or a combination of agents administered to an individual, which improves, in a measurable manner, the differentiation of the epidermal cells, a feature which can be determined, for example, by the indirect immunofluorescence analysis of keratin 10 and involucrin expression and/or by determination of the level of envelope cornified formation [6]. Alternatively, this phrase describes an administered amount of an agent or a combination of agents which can decrease, to a measurable amount, the proliferation of the cells, a feature which can be determined, for example, by measurement of the activity of mitochondrial dehydrogenase enzymes of living cells (MTT assay) [7] and by counting of basal cells level [8].

Preferably, the administration according to the present invention is accomplished by topical application of the agent or the combination of agents.

A currently preferred embodiment of the invention discloses a topical composition comprising the vitamin D compound calcipotriol, in combination with nicotinamide. This composition is especially useful for treating psoriasis.

One embodiment of the present invention includes a topical composition comprising nicotinamide, in combination with 1α,25-dihydroxy-Vitamin D3, which is also referred to herein as “1α,25(OH)₂D3”, and is known as a metabolite of Vitamin D3.

As is described hereinabove, Vitamin D3 and vitamin D compounds are known as useful agents in the treatment of psoriasis and other related skin diseases [6]. However, the synergistic anti-proliferative effect exerted by a Vitamin D compound when used in combination with NA (Nicotinamide) has never been observed hitherto.

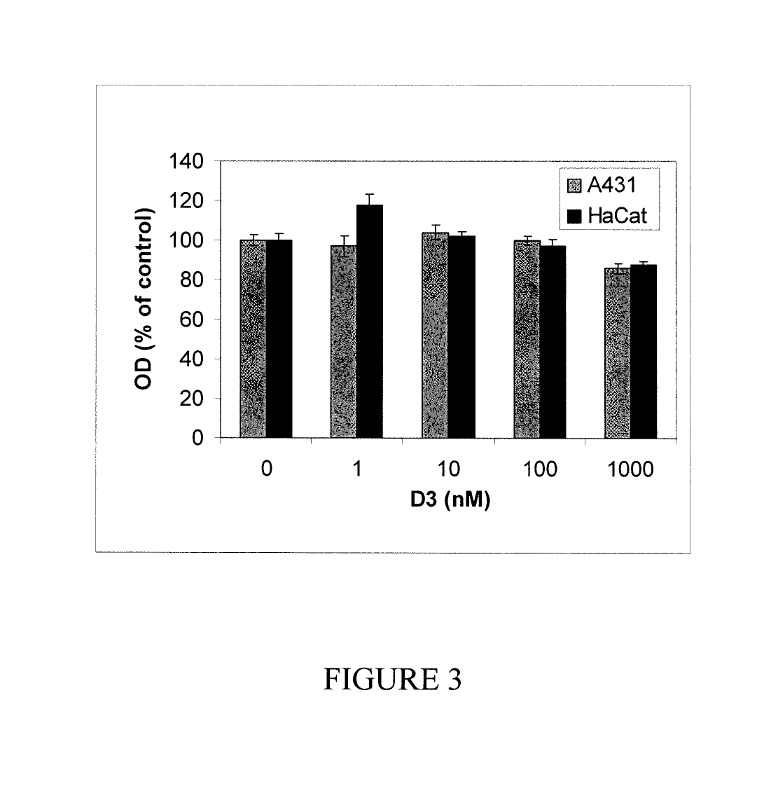

As is described in detail in the Examples section that follows, the above combination was found more effective in promoting differentiation and inhibiting proliferation of different epidermal cell lines, than the sum of each of the individual effects of NA and the vitamin D compound when administered separately. In fact, at the tested concentrations (1 nM-1000 nM), the vitamin D3 metabolite 1α,25-dihydroxy-Vitamin D3 was found inactive toward proliferation and/or differentiation of epidermal cells, as shown and described in relation to FIG. 3.

Non-limiting examples of other suitable vitamin D compounds which can be used in the context of present invention include, without limitation, 25-hydroxycholecalciferol (25 OH D3) and 24R, 25-dihydroxycholecalciferol (24R, 25(OH)₂D3).

For the purpose of convenience, and unless otherwise defined, the term “agent” or “agents” is used hereinafter to describe an active ingredient of the invention, such as a NA (Nicotinamide) agent, or a vitamin D compound, as is defined hereinabove. The phrase “combination of agents” is used hereinafter to describe all the optional combinations of agents that can be used in the context of the present invention, such as a combination of a NA agent and a Vitamin D compound.

As is mentioned in brief hereinabove, while reducing the present invention to practice, it was further surprisingly found that long-term (e.g., 6 months) NA-treated human keratinocytes exhibit high resistance to hydrogen peroxide-induced oxidative stress. These findings demonstrate the capability of a nicotinamide agent to act as a strong antioxidant, which increases the anti-oxidative properties of epidermal cells. This feature further provides a NA agent with the ability to act as an anti-cancer protector of human epidermal cells, as is discussed hereinbelow.

As nicotinamide was found highly active in the treatment of hyperproliferative epidermal pathologies, as is demonstrated herein, it is expected that like nicotinamide, as defined hereinabove, would also exert an anti-oxidative effect on epidermal cells by increasing the anti-oxidative properties of the cells. Hence, according to further aspects of the present invention, there are provided methods of increasing anti-oxidative properties of epidermal cells, preferably human epidermal cells.

Skin cells characterized by increasing sensitivity to oxidative injury, such as cells with predisposition to initiation of tumors can also be treatable by these methods. Increasing the anti-oxidative properties of such cells provides for anti-cancer protection of these cells.

Hence, the methods of increasing anti-oxidative properties of epidermal cells can be efficiently used, for example, in anti-cancer protection of epidermal cells that are relatively susceptible to the oxidative initiation of cancer tumors. As the agents and the combination of agents described hereinabove were found highly active as antiproliferative agents for treating epidermal hyperproliferative pathologies, and as anti-oxidants which increase the anti-oxidative properties of epidermal cells, according to further aspects of the present invention, there are provided compositions which are identified for use in the treatment of benign or malignant hyperproliferative pathologies and/or for use in the treatment of conditions whereby increasing anti-oxidative properties of epidermal cell is advantageous.

When the condition is cancer, increasing the anti-oxidative properties of epidermal cells is highly advantageous as it provides for an anti-cancer protection of the cells.

As used herein, the phrase “anti-cancer protection” describes a condition in which epidermal cells are characterized by increased sensitivity to oxidative injury, such as cells with predisposition to initiation of tumors, and therefore require anti-cancer protection.

In the compositions of the present invention, the concentration of nicotinamide (NA) preferably ranges between 4.1 mM (0.05%) and 491.3 mM (6%), more preferably, between 14.7 mM (0.18%) and 147.4 mM (1.8%). These concentrations are based on the mw=122.13 for nicotinamide.

In other embodiments, the concentration of nicotinamide ranges between about 10 mM and about 160 mM. More preferably, it ranges between about 15 mM and about 130 mM. In certain currently preferred embodiments, it ranges between about 17.2 mM and about 114.6 mM.

A preferred molar ratio between the NA and the Vitamin D compound ranges between about 50:1 and about 6000:1, and a preferred final concentration of the Vitamin D compound typically ranges between 20 μM and 2000 μM, and more preferably between about 72 μM and about 168 These concentrations are based on the molecular weight of Calcipotriol being mw=412.6.

All the pharmaceutical, compositions of the present invention include a pharmaceutically acceptable carrier.

As used herein, the phrase “acceptable carrier” refers to a carrier or a diluent that does not abrogate the biological activity and properties of the applied active agent. When referring to a composition formulated for topical administration, an acceptable carrier or diluent will not cause significant irritation to the skin.

Examples of acceptable carriers that are useful in the context of the present invention for topical administration, include, without limitation, emulsions, creams, aqueous solutions, oils, ointments, pastes, gels, lotions, milks, foams, suspensions and powders.

The carrier of the present invention may include, for example, a thickener, an emollient, an emulsifier, a humectant, a surfactant, a suspending agent, a film forming agent, a foam building agent, a preservative, an antifoaming agent, a fragrance, a lower monoalcoholic polyol, a high boiling point solvent, a propellant, a colorant, a pigment or mixtures thereof.

Therefore, the final composition of the present invention may be, for example, in the form of an oil, a gel, a solid stick, a lotion, a cream, a milk, a foam, a mousse, an aerosol, a spray, an ointment or a fatty ointment and a powder. The compositions of the present invention are preferably topically applied on the treated epidermal cells.

Herein, the term “excipient” describes an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drug agents may be found in “Remington's Pharmaceutical Sciences,” Mack Publishing Co., Easton, Pa., latest edition, which is incorporated herein by reference.

Preferably, the pharmaceutical compositions of the present invention are administered topically.

Pharmaceutical compositions for topical administration are preferably in the form of cream, gel, solution, salve, lotion, ointment or fatty ointment.

Pharmaceutical compositions of the present invention may be manufactured by processes well known in the art, e.g., by means of conventional mixing, dissolving, granulating, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

Compositions for topical administration may include, but are not limited to, lotions, suspensions, ointments gels, creams, drops, liquids, sprays emulsions and powders, as is described hereinabove.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Many of the agents in the claimed compositions of the present invention may be provided as physiologically acceptable salts wherein the agent may form the negatively or the positively charged species. Examples of salts in which the agent forms the positively charged moiety include, without limitation, quaternary ammonium (defined elsewhere herein), salts such as the hydrochloride, sulfate, carbonate, lactate, tartrate, maleate, succinate, etc, wherein the nitrogen of the quaternary ammonium group is a nitrogen of a compound of the present invention which reacts with an appropriate acid. Salts in which the agent forms the negatively charged species include, without limitation, the sodium, potassium, calcium and magnesium salts formed by the reaction of a carboxylic acid group in the molecule with the appropriate base (e.g., sodium hydroxide (NaOH), potassium hydroxide (KOH), calcium hydroxide (Ca(OH)2), etc.).

Determination of a therapeutically effective amount is well within the capability of those skilled in the art, especially in light of the detailed disclosure provided herein.

For any agent or combination of agents used within the scope of the invention, the therapeutically effective amount or dose can be estimated initially from cell culture assays. For example, a dose can be formulated in animal models to achieve a circulating concentration range that includes the IC₅₀ as determined in cell culture (e.g., the concentration of the test compound, which achieves a half-maximal inhibition of the epidermal cells proliferation). Such information can be used to more accurately determine useful doses in humans.

Toxicity and therapeutic efficacy of the agents described herein can be determined by standard pharmaceutical procedures in cell cultures or experimental animals, e.g., by determining the IC₅₀ and the LD₅₀ (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active moiety which are sufficient to maintain the anti-proliferative effects, termed the minimal effective concentration (MEC). The MEC will vary for each preparation, but can be estimated from in vitro data. Dosages necessary to achieve the MEC will depend on individual characteristics and route of administration.

Depending on the severity and responsiveness of the condition to be treated, dosing can also be a single administration of a slow release composition.

The course of treatment can last from several days to several weeks or until cure is effected or diminution of the disease state is achieved.

The amount of an agent or a combination of agents to be administered will, of course, be dependent on the subject being treated, the severity of the affliction, the manner of administration, the judgment of the prescribing physician, etc.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Materials and Experimental Methods

Cell Cultures:

Immortalized human keratinocyte HaCat cells were routinely cultured in 75 cm² flasks using Eagle's minimal essential medium (MEM-EAGLE) supplemented with 5% fetal calf serum (FCS) and 1% antibiotics (penicillin 20 units/ml; streptomycin 20 μg/ml and nystatin 2.5 units/ml) at 37° C. in 95% air/5% CO₂. The medium was replaced every 3-4 days.

Long-term cultures of HaCat cells with NA were obtained by cultivating HaCat cells, for 6 months, in routinely used medium, supplemented with 10 mM NA or 20 mM NA.

Other long-term cultures of cells with other agents are similarly obtained by cultivating HaCat cells, for a prolonged period of time, in routinely used medium supplemented with other agents of the invention, such as: combinations of NA and 1α, 25-dihyroxy-vitamin D3.

Human Epidermal Keratinocytes (passages 3-6), obtained from normal face-lift surgery, were cultivated in serum-free KGM®-2 BulletKit® CC-3107 (Clonetics, USA) medium with low calcium for accelerated proliferation of the keratinocytes.

Epidermal carcinoma A431 cells were cultured in Dulbecco's modified Eagle's medium (DMEM) supplemented with 10% FCS and antibiotics as above.

Reagents:

Nicotinamide (NA); calcitriol (1α, 25-dihyroxy-vitamin D3); calcipotriol, dimethylthiazol-2-yl)-2,5-diphenyltetrazolium bromide (MTT); propidium iodide; dimethylsulphoxide (DMSO); bovine serum albumin (BSA); sucrose; trisodium citrate; igepal CA-630 (NP-40); Tris-(hydroxymethyl)-aminomethane; trypsin; trypsin inhibitor; ribonuclease A; spermin-tetrahydrochloride; sodium dodecylsulfate (SDS); β-mercaptoethanol and hydrogen peroxide (H₂O₂), were all obtained from Sigma (USA).

Eagle's minimal essential medium (MEM-EAGLE); DMEM; antibiotics; fetal calf serum (FCS); L-glutamine; Dulbecco's phosphate buffered saline (PBS); and trypsin 0.05%-EDTA solution were obtained from Biological Industries (Israel).

Keratinocyte Growth Medium®-2 Bullet Kit® CC-3107 (for accelerated proliferation) was received from BioWhittaker, Inc. A Cambrex Company, Clonetics, USA).

Anti-human cytokeratin 10 (NCL-CK10) and involucrin (NCL-INV) mouse monoclonal antibodies were obtained from Novocastra Laboratories Ltd. (UK) and Cy™ 2-conjugated goat anti-mouse IgG was obtained from Jackson Immunoresearch Laboratories, Inc. (USA).

HaCat and A431 cells were propagated in 25 cm² or 75 cm² tissue culture flasks (Corning, USA) and 24-well and 96-well tissue culture plates (Corning, USA) were used for incubation of the cells with different doses of any of the following, alone or in combination: NA (1-50 mM/I), a Vitamin D compound (1-1000 nM).

Proliferation Assays (MTT Method):

The viability and/or proliferation of HaCat and A431 cells and Cultured Human Epidermal Keratinocytes, following treatment with various concentrations of nicotinamide (NA) and various concentrations of a Vitamin D compound, were determined by the MTT assay, according to the procedure described in Mosmann, T: Rapid colorimetric assay for cellular growth and survival: Application to proliferation and cytotoxicity assays, J. Immunol Meth., 65:55-63, (1983), in 96-well microtiter plates.

In brief, an equal number of cells were seeded in each well and incubated for 24 hours. NA or a combination of NA and a Vitamin D compound), at various concentrations, was added thereafter and the wells were incubated for an additional 72 hours. Twenty microliters (20 μl) of 5 mg/ml MTT in phosphate buffered saline (PBS) without Ca⁺² and Mg⁺² were then added to each well. The plates were placed in a CO₂ incubator, and MTT was converted to the insoluble MTT-formazan crystals by mitochondrial dehydrogenases for 3.5 hours. The medium was then removed and the obtained formazan crystals were dissolved in 0.2 ml of DMSO. The amount of formazan was quantified in an ELISA-reader at 550 nm. Background values at 650 nm were subtracted. The data presented encompasses results from three independent experiments.

Differentiation Assays:

Cornified Envelope Formation:

Late differentiation processes in HaCat cells treated with nicotinamide were measured by determining the Cornified cell envelope formation, according to the procedure described in Sun T-T, Green, H: Differentiation of the epidermal keratinocytes in cell culture: formation of cornified envelope, Cell, 9:511-521, 1976.

In brief, cells were seeded in 24-well tissue culture plates and after attachment (24 hours) were exposed to various concentrations of NA (0, 5, 10, 15 and 20 mM) for 96 hours. The cells were thereafter detached and re-suspended in medium. Counting of total and basal (small, rounded) cells was performed using a hemocytometer in tetraplicate aliquots. The remaining cells were spun down, treated with 10 mM Tris-HCl (pH 7.4) supplemented with 1% β-mercaptoethanol and 1% SDS for 10 minutes and cornified envelope cells were counted in tetraplicate aliquots using a hemocytometer. The presented data were results of three independent experiments.

Indirect Immunofluorescence:

Effects of NA on early (keratin k10 expression) and late (involucrin expression) differentiation processes in HaCat cells were estimated by indirect immunofluorescence.

In brief, 2×10⁴ cells/ml were seeded on glass coverslips into Petri dishes with 0, 5, 10 and 20 mM NA. After 72 hours of incubation, cells on the glass coverslips were washed with PBS, fixed by ice-cold mixture of methanol:acetone (1:1) and incubated at −20° C. for 10 minutes. Fixed cells were thereafter washed in PBS and incubated with blocking buffer (1% BSA in PBS) for 10 minutes, to minimize non-specific absorption of the primary antibodies to the coverslips. Thereafter, the cells were incubated for 1 hour with monoclonal antibodies (Keratin 10 expression was detected by antihuman mouse monoclonal antibody, at 1/50 final dilution; Involucrin expression was detected by antihuman involucrin mouse monoclonal antibody at 1/100 final dilution), at 37° C. hour in a humidified chamber. Exhaustive, PBS-washed cells were incubated with Cy™ 2-conjugated goat anti-mouse IgG, at 1/50 final dilution, for 30 minutes at room temperature. The obtained slides were viewed under a Zeiss microscope (Axioskop-2) equipped with epifluorescence optics and the appropriate filters to avoid cross-channel contamination. The level of keratin 10 and involucrin expression was estimated by counting the positive cells relative to the total cell number. In each slide, at least 500-1000 cells were scored. The presented data is a mean of three independent experiments.

DNA Labeling and Flow Cytometry Analysis:

HaCat and A431 cells were seeded in 25 cm² tissue culture flasks and incubated for 72 hours with 0, 5, 10 and 20 mM of NA. Cells treated with 5% ethanol served as positive control of apoptosis. The nuclei for flow cytometry analysis of DNA were prepared by a detergent trypsin method with propidium iodide, according to the procedure described in Lars L Rindelov: A detergent trypsin method for the preparation of nuclei for FACS DNA analysis, Cytometry 3(5)323-327, 1983.

In brief, the cells (10⁶ per tube) were washed with PBS. The cell pellet was re-suspended in 40 μl citrate buffer (pH 7.6) supplemented with 250 mM sucrose, 40 mM trisodium citrate and 5% DMSO. The re-suspended cells were then incubated in 450 μl solution of trypsin (0.15 mg/ml, pH 7.6) for 10 minutes, and thereafter with trypsin inhibitor and ribonuclease A for another 10 minutes. A hundred (100) μg/ml of fluorochrome solution containing propidium iodide were then added to nuclei. The tubes were placed in the dark and the flow cytometry analysis was carried out in fluorescence-activated cell sorter (FACScan; Becton Dickinson, CA). The level of apoptosis was determined using the Cell Quest Program of Becton Dickonson. Each experiment was repeated three times.

Statistical Analysis:

Results are presented as mean±standard deviation of the mean (mean±SD). Statistical significance (P<0.05) was derived by a Student's t-test.

### EXPERIMENTAL RESULTS

### Example 1

### Nicotinamide Inhibits Proliferation of Three Different Epidermal Cell Types

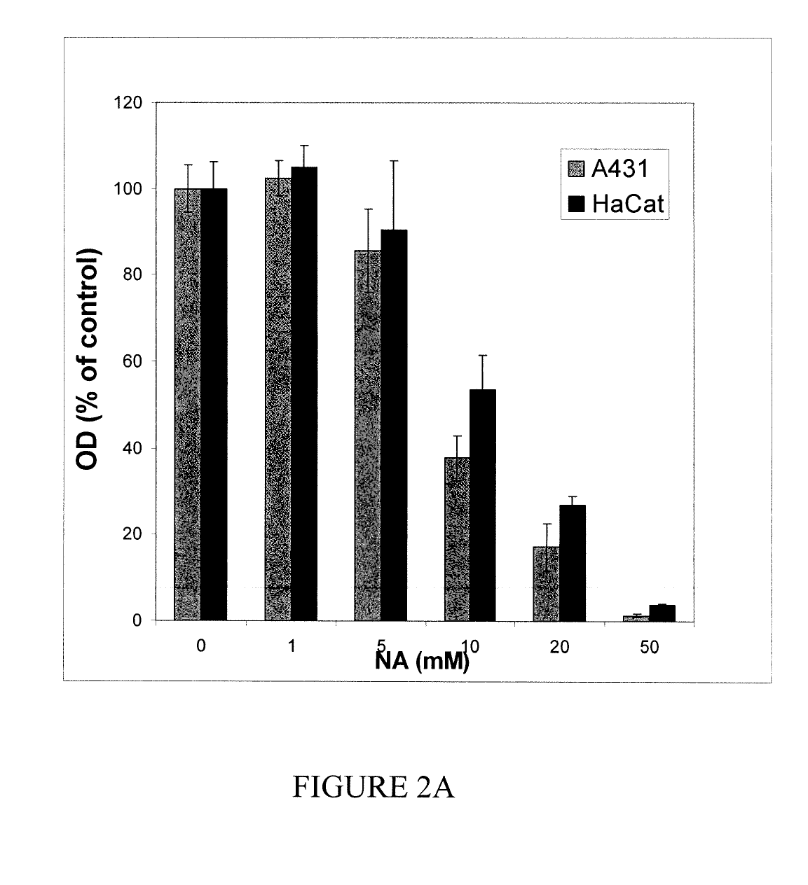

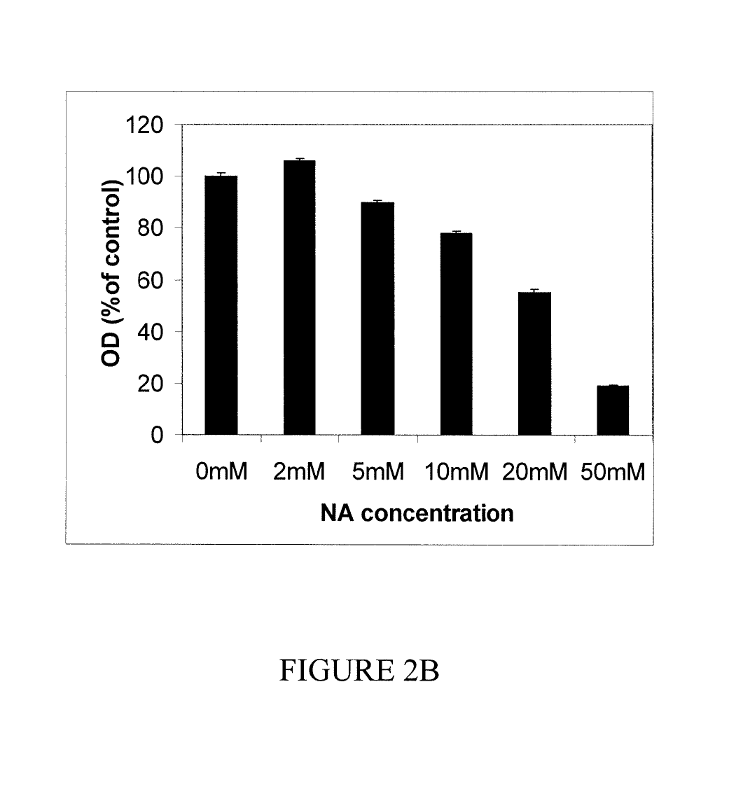

Referring to FIG. 2A, nicotinamide was administered at various doses to cell cultures of immortalized human keratinocyte HaCat cells, or to epithelial carcinoma cells of the human cell line A431. Cell density was 2×10⁴ cells/ml for keratinocyte HaCat cells, and 5×10³ cells/ml for carcinoma A431 cells. Cells were incubated for a period of 72 hours, and the level of proliferation was estimated by the MTT method, described hereinabove. Proliferation was expressed as the percent from control (untreated cells).

Referring to FIG. 2B, nicotinamide was administered at various doses to cultured human epidermal keratinocytes obtained from routine face-lift surgery. Cell density was 2×10⁴ cells/ml. After an incubation period of 72 hours, the level of cell proliferation was estimated by the MTT method, described hereinabove, and was expressed as the percent from control (untreated cells).

As evident from FIGS. 2A and 2B, nicotinamide inhibited proliferation of all three cell types (immortalized keratinocytes, healthy keratinocytes or malignant epidermal cells). Response was similar for all cell types tested, in a dose-dependent manner, with the most effective dose being 50 mM.

### Example 2

### Vitamin D Compound does not Effect Cell Proliferation when Administered Alone (Dosage 1-1000 nM)

HaCat cells and A431 cells, as described hereinabove, were incubated with varying amounts of the Vitamin D compound (1α 25(OH)₂D3) for 72 hours. The obtained results are presented in FIG. 3.

Referring to FIG. 3, the Vitamin D compound did not affect the proliferation of HaCat and A431 cell lines when administered alone, at the tested concentrations.

### Example 3

### Synergistic Effects of NA and a Vitamin D Compound on Cell Proliferation

HaCat cells and A431 cells were incubated with the Vitamin D compound 1α25(OH)₂D3 alone or in combination with nicotinamide, for a period of 72 hours.

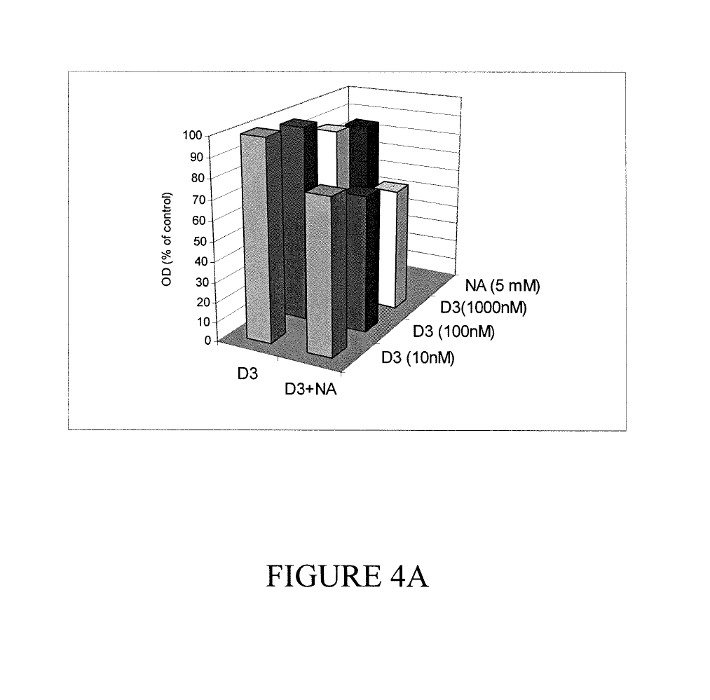

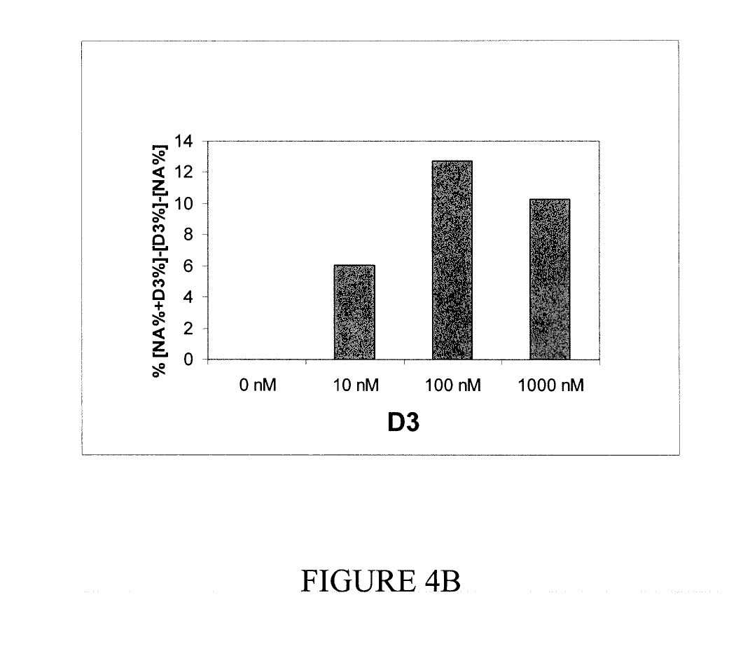

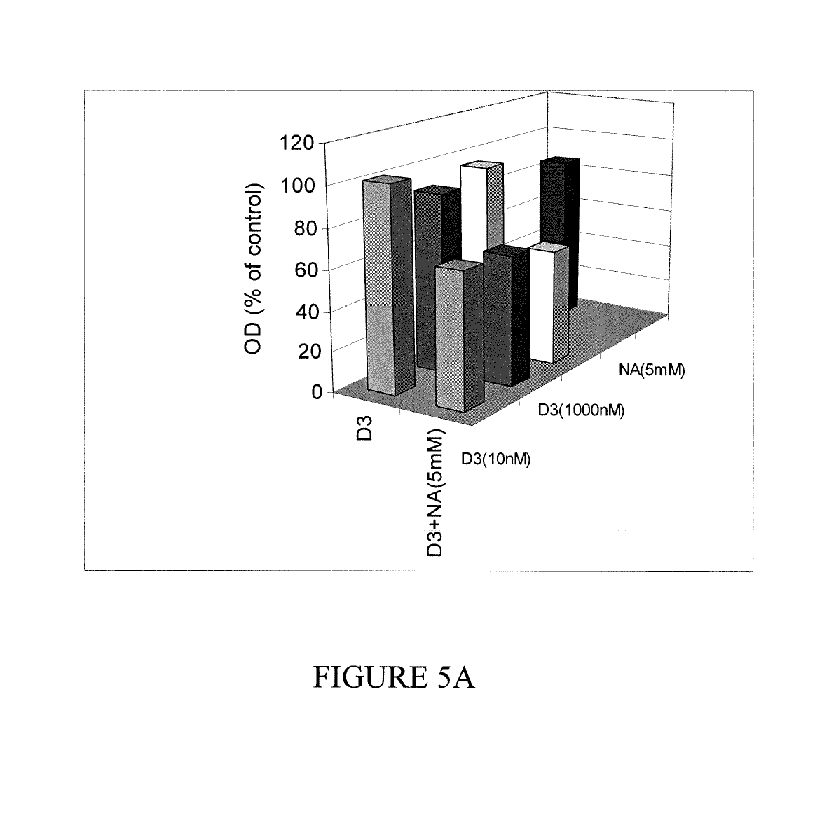

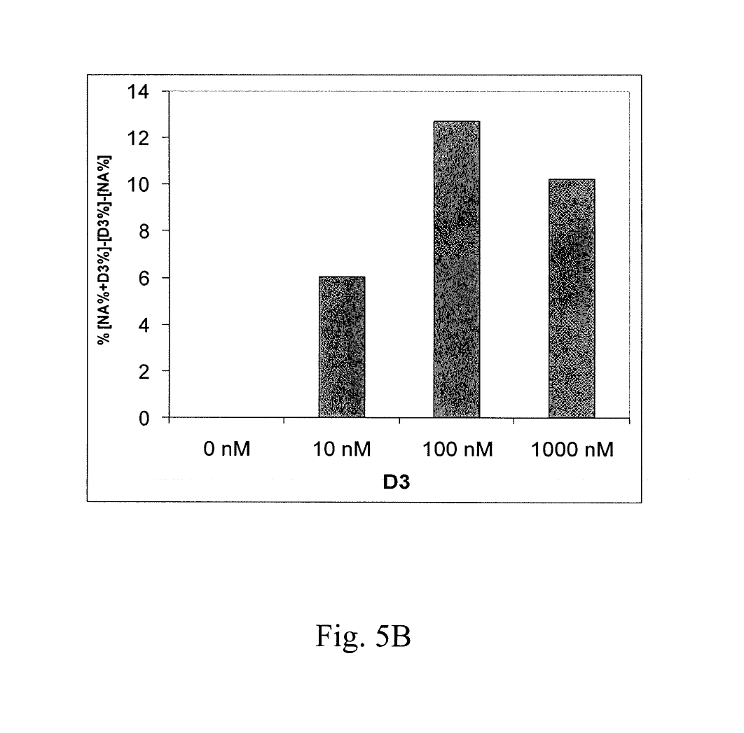

FIGS. 4A and 5A present the results obtained with the above combination in HaCat and A431 cell lines, respectively.

FIGS. 4B and 5B present a deduction of the anti-proliferative effects of 1α25(OH)₂D3 and NA, when applied separately on the cell lines as described hereinabove, from the anti-proliferative effect of the combination of NA and 1α 25(OH)₂D3, presented in FIGS. 4A and 5A. FIG. 4B presents the deduction results in HaCat cells and FIG. 5B presents the deduction results in A431 cells.

As is shown in FIGS. 4B and 5B, the anti-proliferative effect of the combination of NA and 1α25(OH)₂D3 is substantially higher than the summation of the anti-proliferative effects of each of these compounds separately. When a combination of 100 nM 1α25(OH)₂D3 and 5 mM NA was used in HaCat cells, enhancement of 12% was observed in the inhibition of cell proliferation. When a combination of 10 nM of 1α25(OH)₂D3 and 5 mM NA was used in A431 cells, enhancement of 20% was observed in the inhibition of cell proliferation. These results clearly demonstrate the synergistic effect of a combination of NA and a Vitamin D compound in inhibiting epidermal cell proliferation.

### Example 4

### Nicotinamide Promotes Cell Differentiation

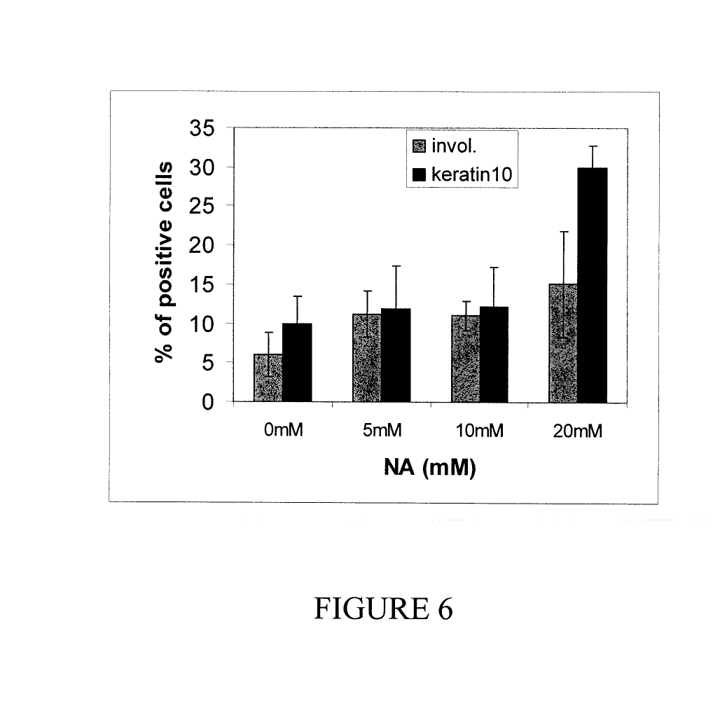

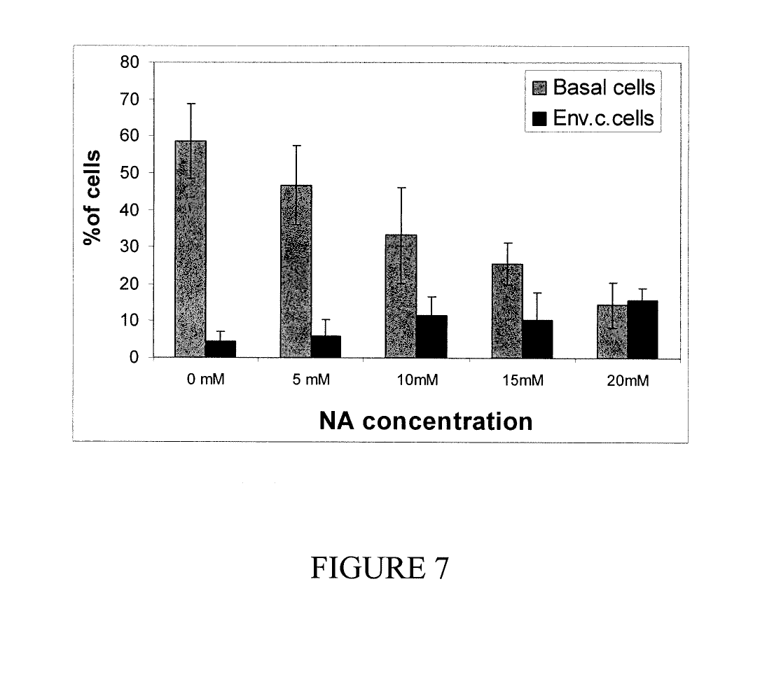

The effect of NA on differentiation was determined by indirect immunofluorescence of keratin K10 and involucrin and by cornified envelope formation, as described above, and the results are presented in FIG. 6 and FIG. 7, respectively.

As is shown in FIG. 6, the NA treatment simulated both expressions of keratin 10 (K10) and involucrin, which are markers of early and late differentiation processes of the epidermal cells, respectively.

As is shown in FIG. 7, the NA treatment also affected the ratio between the amount of cells and envelope cornified cells. A higher proportion of enveloped cornified cells, which are more differentiated cells, in the tested cells was observed.

### Example 5

### Determination of the Apoptotic Dose of Nicotinamide in Cell Culture

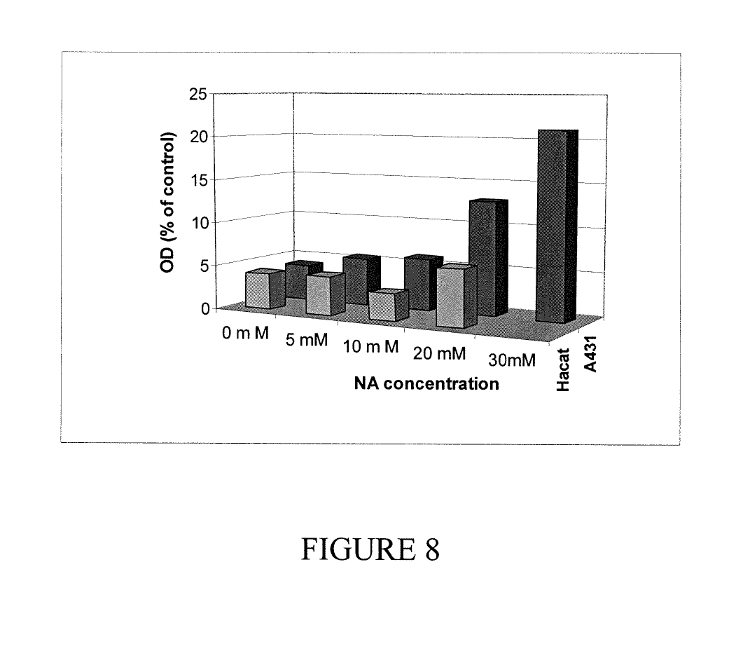

The effect of NA on the level of apoptosis was also determined in HaCat and A431 cells. As is shown in FIG. 8, the determined apoptosis levels show that NA becomes cytotoxic to the cells at a concentration of 30 mM in A431 cells and at a concentration of 50 mM in HaCat cells. These results are significant since they demonstrate that the effect of NA on cell proliferation, as is expressed, for example, in FIG. 2, is effectively exerted by NA concentrations that are lower than the cytotoxic level of NA, namely, at concentrations lower than the concentrations that are toxic to cells.

### Example 6

### Resistance of HaCat Cells Long-Term Cultured with NA (10 mM) to Hydrogen Peroxide-Induced Oxidative Stress

2×10⁴ cells/ml immortalized human keratinocyte HaCat cells cultivated routinely, or the same amount of HaCat cells cultured with 10 mM NA during 6 months, as is described hereinabove, were incubated with increasing concentrations of hydrogen peroxide for 24 hours. The cytotoxicity was estimated by the MTT method described above and was expressed as the percent from control (untreated cells).

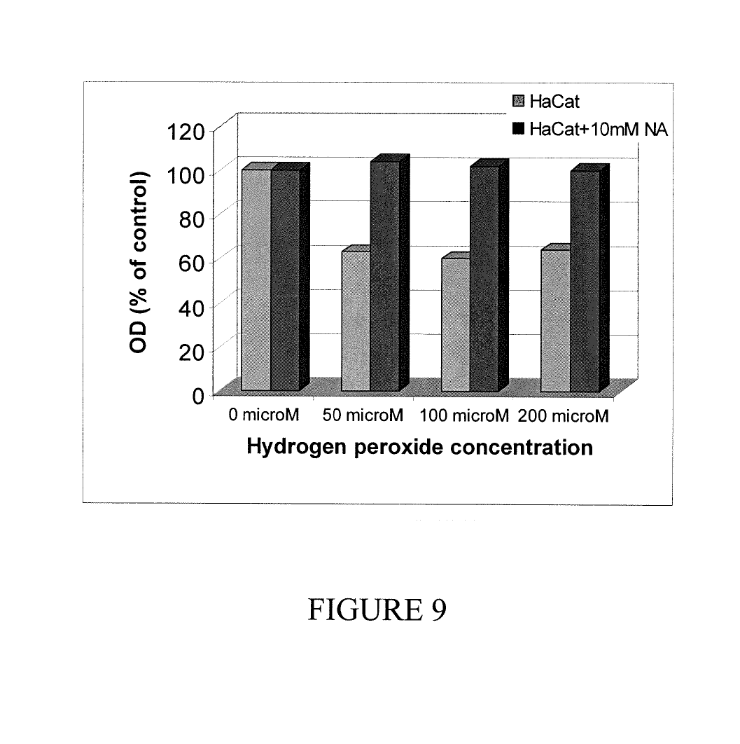

The obtained results are presented in FIG. 9, which demonstrates that while HaCat cells that were cultivated routinely (without NA supplementation) were significantly injured by hydrogen peroxide, the HaCat cells long-term cultured with NA remained unaffected. These data indicate that long-term treatment with NA increases the anti-oxidative properties of human epidermal cells.

### Example 7

### Synergistic Effect of Calcipotriol and NA in Animal Model

In vivo studies were performed to measure the antipsoriatic activity (potency) of compositions comprising NA and calcipotriol, utilizing a standard mouse-tail test. The induction of a granular layer (orthokeratosis) in scale areas of mouse-tail skin is a well-known relevant parameter for anti-psoriatic activity. A high ratio of orthokeratosis and parakeratosis (abnormal maturation), is indicative for high anti-psoriatic activity.

Mice—Male albino mice (ICR) weighing 25-27 g were used, with 6 mice per formulation and 6 mice for the control.

Treatments: 6 treatments per week for 7 days

Treatment: Topical application once a day on the tail base: about 1 cm from the proximal end of the tail to about 2.5 cm section long, for 3.5 hours. The treatment area was pre-rinsed with saline. During the treatment, the tail was protected from licking by mounting collars on the mouse chest.

At the conclusion of the experiment the mice were sacrificed. The treated section of tail, approximately 2.5 cm in length, was removed and fixed in 4% formalin Longitudinal histological sections from the treated tail were prepared by pathologists. 10 sections per tail were made.

An evaluation of the level (%) of orthokeratosis on the scaly tail areas was performed by a dermatological laboratory.

The extensive test was performed with microscopic evaluation of 90-100 scale areas evaluated per each treatment group.

Treatments were as follows:
1. Placebo (PL)
2. Calcipotriol (CP) 50 μg/gr (0.005%),
3. Nicotinamide (NA) 2.1 mg/gr (0.21%)
4. Calcipotriol and Nicotinamide in combination (CPNA), with the following concentration of actives: (NA) 2.1 mg/gr (0.21%)+(CP) 50 μg/gr (0.005%).
   All mice participating were randomized before evaluation and treatments were reassigned to data points only after microscopic analysis was complete in a fully blinded procedure.

Treatments were analyzed statistically, using analysis of variance (ANOVA) calculations for all treatments.

Results

Treatment with either CP (calcipotriol) or NA (nicotinamide) provided visually detectable antipsoriatic changes in mouse tail scales, however treatment with the combination CPNA (calcipotriol+nicotinamide) was superior by far, indicating a synergistic effect for the combination of CP and NA. The mean % Orthokeratosis/Scale per treatment and their standard deviations are presented in Table 1.

TABLE 1

| | % Orthokeratosis/ |
|---|---|
| Formulation | Scale ± SD |
| Placebo (PL) (Vehicle) | 30.3 ± 4.4 |
| Calcipotriol (CP) 50 μg/g | 44.5 ± 6.1 |
| Nicotinamide (NA) 2.1 mg/g | 46.5 ± 9.2 |
| Combined (CP + NA) | 96.0 ± 10.6 |

The synergistic index SI of the combined treatment (CPNA) was calculated by dividing the measured results of the combination, by the sum of the separate treatments. All treatments are first adjusted by subtracting the placebo result from each of the separate results, as in the following equation:

SI=(CPNA−PL)/{[(CP−PL)+(NA−PL)}

SI=(96.0−30.3)/[(44.5−30.3)+(46.5−30.3)]=2.16

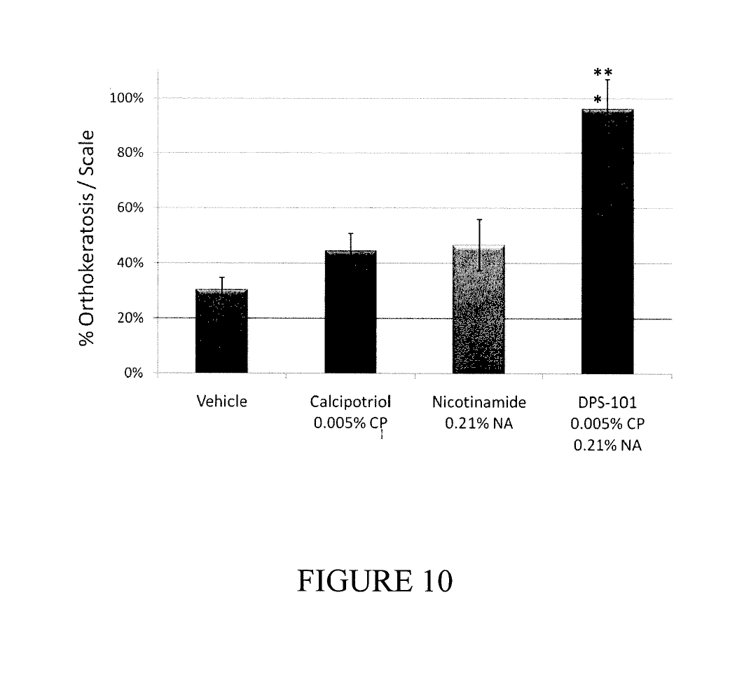

The value SI was calculated to be 2.16 It is therefore clear that synergism exists between CP and NA for the conditions of the performed test.
FIG. 10 presents a graph depicting the results of the above analysis. The synergistic effect is evident for the combination of calcipotriol and nicotinamide (CP+NA) at the above-mentioned concentrations. The synergism is statistically significant (p<0.0001) as compared to the monotherapies of NA and CP.

### Example 8

### Synergistic Effect of Calcipotriol and NA in Human Trials

A Phase IIB dose ranging trial was performed on 168 patients suffering from bilateral psoriatic lesions, with two different treatment regimens (arms) per patient. In total, 336 treatments were divided into a 7-Arm trial (N=48 per arm).

The trial arms were:

1. Placebo

2. Nicotinamide monotherapy

3. Calcipotriol monotherapy

4. Four Combinations of Nicotinamide and Calcipotriol, with the nicotinamide present at one of four predetermined concentrations, and the Calcipotriol present at a constant concentration.

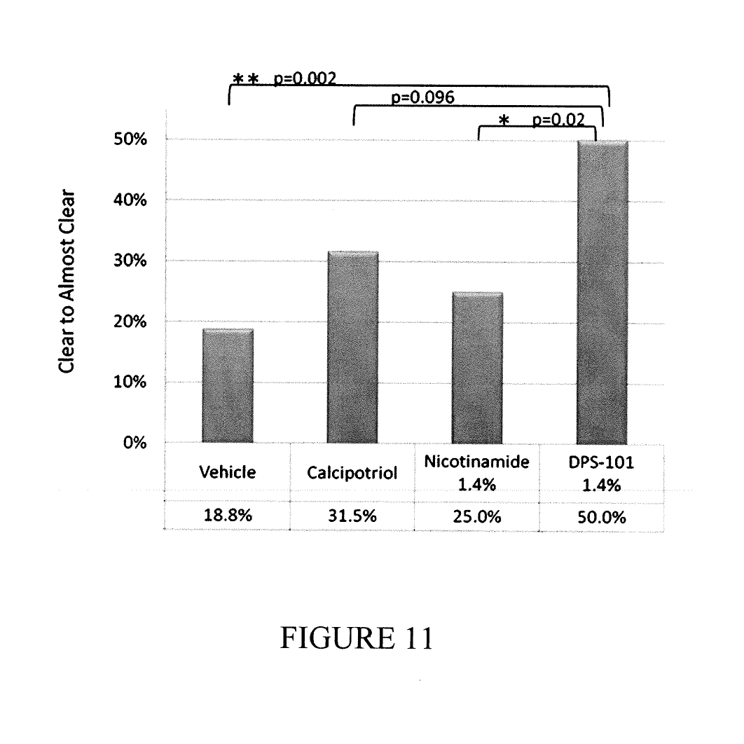

The Phase IIB trial was multicenter, randomized and double-blinded. Patients were treated by topical application of the predetermined composition twice daily, morning and night, for 12 weeks.
Efficacy was measured using the 13-point TLPSS score, (rated on a scale of 0 to 12), which is produced by summation of three partial scores: Scaling (0-4), Erythema (0-4) and Plaque Elevation (0-4).
A subset of TLPSS 0 to 2 which is similar to the definition of “Clear to Almost Clear” of disease symptoms was used as a successful end point.
The DPS101 composition, containing 1.4% Nicotinamide, in combination with calcipotriol, was shown to be the most potent combination.
Referring to FIG. 11, the results illustrate that about 50% of patients treated with the combination of Calcipotriol and Nicotinamide DPS 101, reached the end point of “Clear to Almost Clear” of disease symptoms. In comparison, only 25%-31.5% of patients treated with Calcipotriol monotherapy or Nicotinamide monotherapy, reached this end point.
Statistically significant efficacy was evident relative to placebo (p=0.002) and relative to nicotinamide monotherapy (p=0.02). A trend was evident towards significance vs. calcipotriol monotherapy (p=0.096) even in such small cohorts of 48 patients per arm.

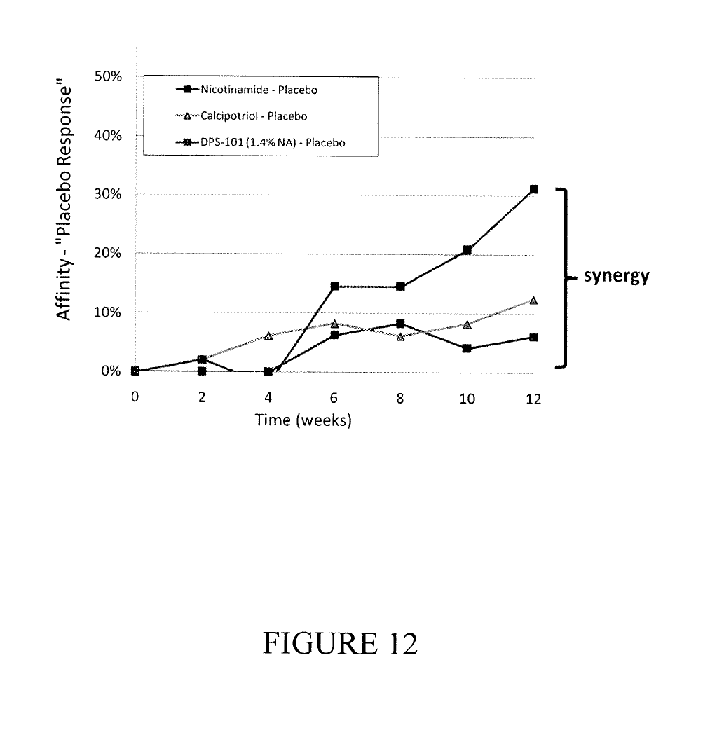

Referring to FIG. 12, the synergistic character of DPS-101 is highlighted when “neutralizing” the placebo response by subtracting the placebo-arm (vehicle) from each of the treatment arms.

The invention discloses that administration of topical compositions comprising calcipotriol and nicotinamide, is superior for treating psoriasis, when compared to administration of only one of these ingredients. In summary, the invention thus demonstrates that the novel combination of a vitamin D compound such as Calcipotriol and nicotinamide, acts in synergism to alleviate symptoms of hyperproliferative diseases, such as psoriasis.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED BY NUMERALS

1. Morimoto S., Yoshikawa, K., Konzuka, T., et al. “An open study of vitamin D3 treatment in psoriasis vulgaris”, Br. J. Dermatol., 115:421-429, (1986).
2. Ockenfels, H. M., Nuβbaum, G., Schultewolter, T., Burger, P M., Goos, M.: “Cyclosporin A, FK506 and dithranol alter tyrosine-specific protein phosphorylation in HaCat keratinocytes”. Arch. Dermatol. Res., 287:304-309, (1995).
3. Paramio, J. M., and Jorcano, J. L.: “Role of protein kinases in the in vitro differentiation of human HaCat cells”. Brit. J. Dermatol., 137:44-50, (1997)).
4. Ahmad, N., Feyes, D. K., Agarwal, R., Mukhtar, H: Photodynamic therapy results in induction of WAF1/CIPI/P21 leading to cell cycle arrest and apoptosis. Proc. Natl. Acad. Sci. USA, 95:6977-6982, (1998).
5. Nikoloff, B. J., Fisher, G. J., Mitra, R. S., Voorhees, J. J.: “Additive and Synergistic Antiproliferative Effect of Cyclosporin A and Gamma Interferon on Cultured Human Keratinocytes”. Amer. J. Pharmacol., 131:12-18, (1988).
6. Sun T-T, Green, H: Differentiation of the epidermal keratinocyte in cell culture: formation of cornified envelope, Cell, 9:511-521, (1976).
7. Mosmann, T: Rapid colorimetric assay for cellular growth and survival: Application to proliferation and cytotoxicity assays. J. Immunol. Meth., 65:55-63, (1983).
8. Sun T-T, Green, H: Differentiation of the epidermal keratinocyte in cell culture: formation of cornified envelope (Cell, 9:511-521 (1976).

What is claimed is:

1. A method of treating a benign or malignant hyperproliferative epidermal pathology in a subject in need thereof, which comprises: topically administering to the skin of the subject a composition comprising therapeutically effective amounts of the combination of:
a) a vitamin B3 derivative selected from nicotinamide, and a nicotinamide derivative; and
b) a vitamin D compound; and
c) optionally, a pharmaceutically acceptable carrier;
wherein the hyperproliferative benign epidermal disease to be treated is psoriasis, ichythyiosis, common warts, keratoacanthoma, seborrhoic keratosis or seborrhea.

2. The method of claim 1, wherein the hyperproliferative malignant epidermal disease to be treated is squamous-cell carcinoma (SCC), basal cell carcinoma (BCC) or a non-melanoma skin cancer (NMSC).

3. The method of claim 1, wherein the vitamin D compound is a vitamin D metabolite, a vitamin D agonist, a vitamin D prodrug, a vitamin D derivative, or a vitamin D analog.

4. The method of claim 1, wherein the vitamin D compound is vitamin D3, vitamin D2, 25(OH)D3, 1α,25(OH)2D3, a 19-nor-vitamin D compound, a cyclopentano-vitamin D derivative, a trihydroxycalciferol derivative, a 20-methyl substituted vitamin D derivative, a 23-oxa-derivative, or a hydroxylated 24-homo-vitamin D derivative compound.

5. The method of claim 1, wherein the vitamin D compound is calcipotriol and the vitamin B3 derivative is nicotinamide.

6. The method of claim 1, wherein the vitamin D compound is calcipotriol present at a concentration of 0.001% to 0.01%.

7. The method of claim 1, wherein the vitamin B3 derivative is nicotinamide present at a concentration of 0.05% to 6% of the final composition.

8. The method of claim 7, wherein the nicotinamide and the Vitamin D compound are present at a molar ratio of between 50:1 and 6000:1.

9. The method of claim 8, wherein the concentration of nicotinamide is 1.4% of the final composition, and the vitamin D compound is calcipotriol present at a concentration of 0.005% of the final composition.

10. A method for inhibiting the proliferation or differentiation of epidermal cells or for increasing the anti-oxidative properties of epidermal cells which comprises topically administering to a subject in need, therapeutically effective amounts of a composition comprising a combination of the following active ingredients:
a) a vitamin B3 derivative selected from: nicotinamide, and a nicotinamide derivative in an amount effective to act as an anti-oxidant; and
b) a vitamin D compound; and
c) optionally, a pharmaceutically acceptable carrier.

11. The method of claim 10, wherein the vitamin D compound is calcipotriol present at a concentration of 0.001% to 0.01%.

12. The method of claim 10, wherein the vitamin B3 derivative is nicotinamide present at a concentration of 0.05% to 6% of the final composition.

13. The method of claim 10, wherein the nicotinamide and the Vitamin D compound are present at a molar ratio of between 50:1 and 6000:1.

14. The method of claim 13, wherein the vitamin B3 derivative is nicotinamide and the Vitamin D compound is calcipotriol.
